(19) United States
(12) Patent Application Publication
(10) Pub. No.: US 2026/0170641 A1
(43) Pub. Date: Jun. 18, 2026

(54) INFORMATION PROCESSING DEVICE, INFORMATION PROCESSING METHOD, AND COMPUTER-READABLE RECORDING MEDIUM

(71) Applicant: DEEPEYEVISION INC., Shimotsuke-shi, Tochigi (JP)

(72) Inventors: Hidenori TAKAHASHI, Shimotsuke-shi, Tochigi (JP); Yusuke KONDO, Shimotsuke-shi, Tochigi (JP)

(21) Appl. No.: 19/125,093

(22) PCT Filed: Dec. 7, 2023
(86) PCT No.: PCT/JP2023/043769

§ 371 Date: Apr. 28, 2025

(30) Foreign Application Priority Data
Dec. 12, 2022 (JP) 2022-197888

(51) Int. Cl. G06T 7/00; A61B 3/14; G06T 11/00

(52) U.S. Cl. CPC G06T 7/0012; A61B 3/14; G06T 11/00; G06T 2207/30041; G06T 2207/30101

(57) ABSTRACT

An information processing device, an information processing method, and a computer-readable recording medium capable of obtaining a measurement value indicating an evaluation of features of the whole tissues of a portion to be examined in an image are provided.

An information processing device 20 includes an image acquisition unit 224 that acquires a first image acquired by imaging of a portion to be examined of a subject, a first classification unit 226 that acquires a confidence score that a first attribute of the portion to be examined of the first image is normal and/or a confidence score that the first attribute of the portion to be examined of the first image has a certain deviation degree from the normal first attribute, and an output unit 2210 that outputs an inference result based on the confidence score acquired by the first classification unit.

### CROSS-REFERENCE TO RELATED APPLICATION

This application claims priority to Japanese Patent Application No. 2022-197888 filed on Dec. 12, 2022, which is incorporated herein by reference in its entirety.

### TECHNICAL FIELD

The present disclosure relates to an information processing device, an information processing method, and a computer-readable recording medium.

### BACKGROUND ART

A fundus is a portion where blood vessels and a ganglionic layer can be directly observed. A fundus image acquired using a fundus camera, or the like, includes information for finding diseases of the eyeball, diabetes, hypertension, and arteriosclerosis or evaluating degrees of progress thereof.

### CITATION LIST

### Patent Literature

Patent Literature 1: Japanese Patent Laid-Open No. 2014-193225

### SUMMARY OF INVENTION

### Technical Problem

Patent Literature 1 discloses a fundus image processing device that calculates an artery-vein diameter ratio at a fundus of a subject eye by processing a fundus image of the subject eye, the fundus image processing device including papilla identification means for identifying a papilla included in the fundus image, blood vessel identification means for identifying blood vessels included in the fundus image, upward diameter ratio calculation means for calculating an upward diameter ratio which is a diameter ratio between blood vessels positioned in an upper region from a height of the papilla identified by the papilla identification means, among the blood vessels identified by the blood vessel identification means, and downward diameter ratio calculation means for calculating a downward diameter ratio which is a diameter ratio between blood vessels positioned in a lower region from the height of the papilla among the blood vessels identified by the blood vessel identification means.

According to the fundus image processing device disclosed in Patent Literature 1, it is possible to calculate an artery-vein diameter ratio with certain accuracy. On the other hand, with the fundus image processing device disclosed in Patent Literature 1, individual measurement results are calculated while attention is focused on individual blood vessels in a process of calculating the artery-vein diameter ratio, and thus, in a case where arteries and veins are detected from a wide region of the fundus image, enormous processing is performed, which requires a large amount of computer resources.

It is therefore an object of the present disclosure to provide an information processing device, an information processing method, and a computer-readable recording medium capable of obtaining a measurement value indicating an evaluation of features of the whole tissues at a portion to be examined in an image.

### Solution to Problem

An information processing device according to one aspect of the present disclosure includes an image acquisition unit that acquires a first image acquired by imaging of a portion to be examined of a subject, a first classification unit that acquires a confidence score that a first attribute of the portion to be examined of the first image is normal and/or a confidence score that the first attribute of the portion to be examined of the first image has a certain deviation degree from the normal first attribute, and an output unit that outputs an inference result based on the confidence score acquired by the first classification unit.

According to this aspect, the information processing device can obtain a measurement value indicating an evaluation of features of the whole first attribute of the portion to be examined of the first image using the first classification unit that acquires the confidence score that the first attribute of the portion to be examined of the first image is normal and/or the confidence score that the first attribute of the portion to be examined of the first image has a certain deviation degree from the normal first attribute. Compared to a case where individual attributes of the portion to be examined of the first image are measured and evaluated, the information processing device can obtain the measurement value with fewer computer resources.

The above-described information processing device may further include a second classification unit that acquires a confidence score that a second attribute of the portion to be examined of the first image is normal and/or a confidence score that the second attribute of the portion to be examined of the first image has a certain deviation degree from the normal second attribute, and the output unit may output the inference result based on the confidence score acquired by the second classification unit. According to this aspect, the information processing device can obtain a measurement value indicating an evaluation of features of the whole attribute also for the second attribute in addition to the first attribute for the portion to be examined of the first image.

In the above-described information processing device, the first image may be a fundus image of the subject, and the first attribute and the second attribute may be attributes regarding an artery. According to this aspect, the information processing device can obtain a measurement value indicating an evaluation of features of the whole first attribute and second attribute regarding the artery of the fundus image.

The above-described information processing device may further include a third classification unit that acquires a confidence score that a third attribute of the portion to be examined of the first image is normal and/or a confidence score that the third attribute of the portion to be examined of the first image has a certain deviation degree from the normal third attribute, and the output unit may output the inference result based on the confidence score acquired by the third classification unit.

According to this aspect, the information processing device can obtain a measurement value indicating an evaluation of features of the whole attribute also for the third attribute in addition to the first attribute and the second attribute for the portion to be examined of the first image.

In the above-described information processing device, the first image may be a fundus image of the subject, the first attribute and the second attribute are attributes regarding an artery, and the third attribute may be an attribute regarding a fundus. According to this aspect, the information processing device can obtain a measurement value indicating an evaluation of features of the whole third attribute regarding the fundus in addition to the features of the whole first attribute and second attribute regarding the artery of the fundus image.

In the above-described information processing device, the first classification unit, the second classification unit, and the third classification unit may be a single classification unit. According to this aspect, the information processing device can obtain a measurement value indicating an evaluation of features of the whole three attributes while memory capacity required for the classification units is reduced.

In the above-described information processing device, the second classification unit may acquire the confidence score that the second attribute of the portion to be examined is normal and/or the confidence score that the second attribute of the portion to be examined has a certain deviation degree from the normal second attribute by inputting the first image to a second inference model, and the second inference model may be a model that estimates whether the second attribute of the portion to be examined included in the first image is normal or has a certain deviation degree from the normal second attribute. According to this aspect, the information processing device can obtain a measurement value indicating an evaluation of features of the whole second attribute of the portion to be examined using the inference model that has learned the normal second attribute and/or features that deviate from the normal second attribute from an image of a healthy portion to be examined and an image of an unhealthy portion to be examined.

In the above-described information processing device, the first classification unit may acquire the confidence score that the first attribute of the portion to be examined is normal and/or the confidence score that the first attribute of the portion to be examined has a certain deviation degree from the normal first attribute by inputting the first image to a first inference model, and the first inference model may be a model that estimates whether the first attribute of the portion to be examined included in the first image is normal or has a certain deviation degree from the normal first attribute. According to this aspect, the information processing device can obtain a measurement value indicating an evaluation of features of the whole second attribute of the portion to be examined using an inference model that has learned the normal second attribute and/or features that deviate from the normal second attribute from an image of a healthy portion to be examined and an image of an unhealthy portion to be examined.

The above-described information processing device may further include an image creation unit that creates a second image in which a region that contributes to classification by the first classification unit is visualized, and the output unit may output the inference result based on the second image created by the image creation unit. According to this aspect, the information processing device can grasp a region in which deviation from the normal first attribute is anticipated in the portion to be examined of the first image.

In the above-described information processing device, the image creation unit may create the second image by adjusting a value representative of a degree of contribution to the classification by the first classification unit based on a confidence score of the classification by the first classification unit. According to this aspect, the information processing device can point out, with high accuracy, a region in which deviation from the normal first attribute is anticipated.

In the above-described information processing device, the first classification unit may acquire a confidence score that the first attribute of the portion to be examined is normal and a plurality of confidence scores that the first attribute of the portion to be examined have certain deviation degrees from the normal first attribute. According to this aspect, the information processing device can obtain confidence scores regarding a plurality of deviation degrees with different deviation degrees from the normal first attribute.

A method according to another aspect of the present disclosure includes acquiring a first image acquired by imaging of a portion to be examined of a subject, acquiring a confidence score that a first attribute of the portion to be examined of the first image is normal and/or a confidence score that the first attribute of the portion to be examined of the first image has a certain deviation degree from the normal first attribute, and outputting an inference result based on the confidence score.

A computer-readable recording medium according to still another aspect of the present disclosure records a program that causes one or more computers to execute processing of acquiring a first image acquired by imaging of a portion to be examined of a subject, processing of acquiring a confidence score that a first attribute of the portion to be examined of the first image is normal and/or a confidence score that the first attribute of the portion to be examined of the first image has a certain deviation degree from the normal first attribute, and processing of outputting an inference result based on the confidence score.

An information processing device according to yet another aspect of the present disclosure includes a learning unit that causes a machine learning model to learn by inputting, to the machine learning model, an image for which information indicating healthy or a deviation degree from a normal first attribute is labeled as ground truth data, and a model output unit that outputs the learned model learned at the learning unit. According to this aspect, the information processing device can obtain the learned model that can be used in inference of the normal first attribute and/or the deviation degree from the normal first attribute.

### Advantageous Effects of Invention

According to the present disclosure, it is possible to provide an information processing device, an information processing method, and a computer-readable recording medium capable of obtaining a measurement value indicating an evaluation of features of the whole tissues of a portion to be examined in an image.

### BRIEF DESCRIPTION OF DRAWINGS

FIG. 1 is a view illustrating a network configuration of an information processing system according to an embodiment.

FIG. 2 is a schematic view for explaining processing by a learning device according to an embodiment.

FIG. 3 is a schematic view for explaining processing by an inference device according to an embodiment.

FIG. 4 is a block diagram of the learning device according to an embodiment.

FIG. 5 is a block diagram of the inference device according to an embodiment.

FIG. 6 is a flowchart indicating learning processing by the learning device according to an embodiment.

FIG. 7 is a flowchart indicating inference processing by the inference device according to an embodiment.

FIG. 8 is a view indicating a function to be used for adjusting density of a heat map according to an embodiment.

FIG. 9 is a view illustrating a screen example that presents the heat map according to an embodiment.

### DESCRIPTION OF EMBODIMENTS

An embodiment of the present invention will be described with reference to the accompanying drawings. Note that the following embodiment is provided to facilitate understanding of the present invention and is not intended to limit the present invention. Further, the present invention can be modified in various manners without deviating from the gist of the present invention. Still further, a person skilled in the art could employ embodiments in which respective elements described below are replaced with equivalents, and such embodiments are also included in the scope of the present invention.

### System Configuration

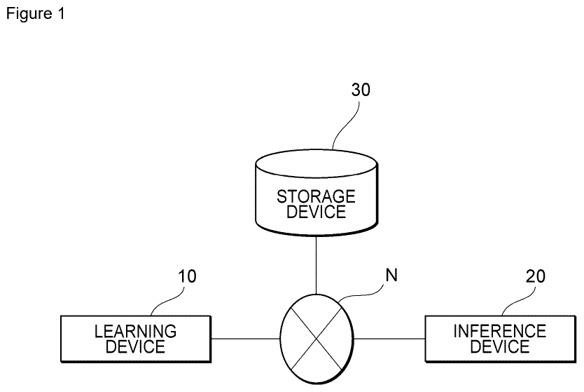

Outline of the present disclosure will be described using FIGS. 1, 2 and 3.

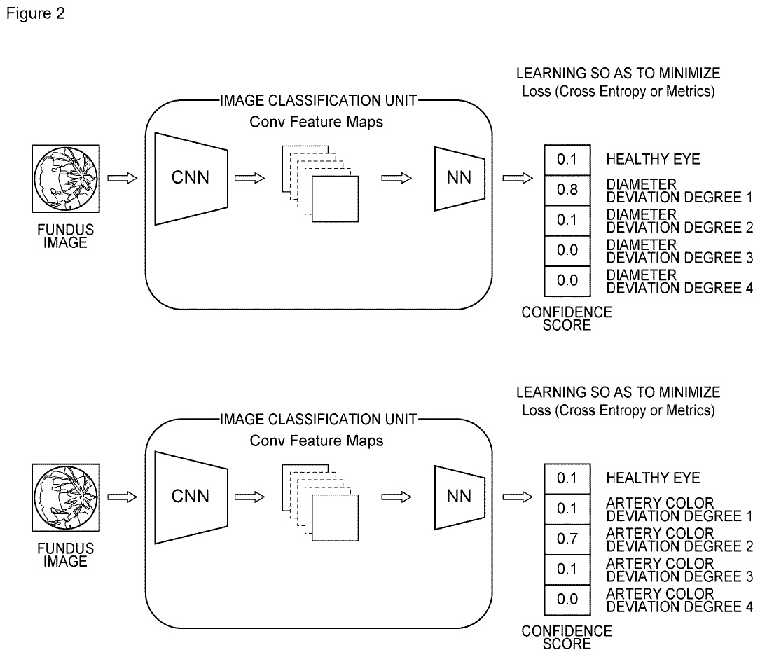

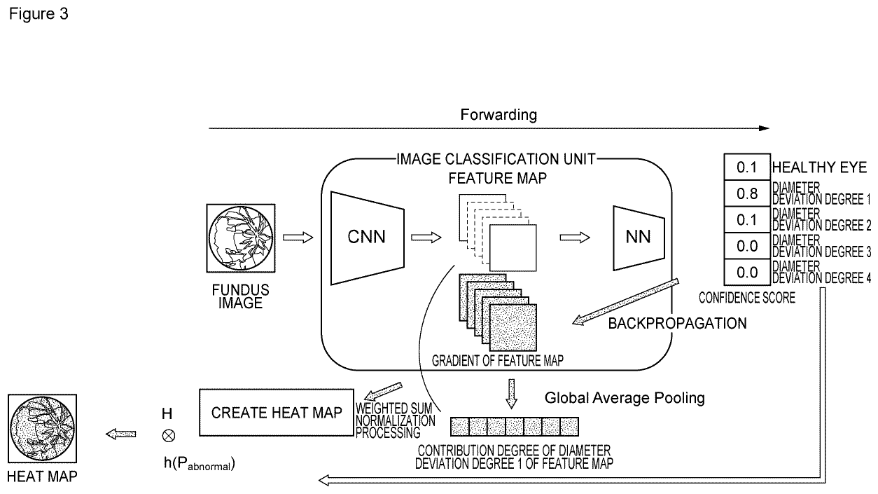

FIG. 1 is a view illustrating a network configuration of an information processing system according to an embodiment. FIG. 2 is a schematic view for explaining processing by a learning device according to an embodiment. FIG. 3 is a schematic view for explaining processing by an inference device according to an embodiment.

The information processing system includes a learning device 10, an inference device 20, and a storage device 30. The learning device 10 is connected to the inference device 20 and the storage device 30 via a communication network N. The communication network N may be either a wired communication network or a wireless communication network constituted with a wired or wireless line and may be the Internet or a local area network (LAN).

The learning device 10 performs learning of a machine learning model based on learning data stored in the storage device 30 and stores the learned model in the storage device 30. While the learning device 10 according to the present embodiment includes the machine learning model, the machine learning model may be provided in a device different from the learning device 10.

Here, the machine learning model is a model which has a certain model structure and a processing parameter that fluctuates by learning processing and for which identification accuracy is improved by the processing parameter being optimized based on experience obtained from the learning data. In other words, the machine learning model is a model that learns an optimal processing parameter through the learning processing. While as an algorithm of the machine learning model, for example, support vector machine, logistic regression, a neural network, or the like, can be used, the type of the algorithm is not particularly limited. The machine learning model that performs the learning also includes a model before learning, and a model that has already performed some kind of learning by the learning data.

Note that the learned model is a model that has performed learning in advance using appropriate learning data for the machine learning model by an arbitrary machine learning algorithm. However, the learned model is not a model that will not perform further learning and can perform additional learning.

The inference device 20 outputs output data in accordance with features of input data using the learned model. The inference device 20 according to the present embodiment performs inference using the learned model acquired from the storage device 30. Here, acquisition of the learned model refers to acquisition of information necessary for reproducing functions of the learned model at the inference device 20. For example, in a case where a neural network is used as the machine learning model, acquisition of the learned model refers to acquisition of information regarding at least the number of layers of the neural network, the number of nodes regarding each layer, a weight parameter of a link connecting between the nodes, a bias parameter regarding each node, and a function form of an activating function regarding each node.

The storage device 30 stores learning data to be used for learning of the machine learning model. The storage device 30 according to the present embodiment stores, as first learning data, a fundus image for which information indicating “healthy” or a deviation degree from a normal artery diameter is labelled as ground truth data. For example, if hyperpiesia progresses, blood vessels throughout the body are strained, and capillary blood vessels are damaged at the fundus, which causes constriction of the blood vessels. While in the present embodiment, a total of four labels from a diameter deviation degree 1 with high similarity to a healthy eye to a diameter deviation degree 4 with low similarity to a healthy eye are used as information indicating the deviation degree from the normal artery diameter, the deviation degree from a normal artery diameter may be indicated using other arbitrary numbers of labels.

Further, the storage device 30 stores, as second learning data, a fundus image for which information indicating “healthy” or a deviation degree from normal artery color is labeled as ground truth data. For example, while normal artery color is red, if arteriosclerosis progresses, walls of the blood vessels become thick, and the center of artery looks whitish. While in the present embodiment, a total of four labels from an artery color deviation degree 1 with high similarity to a healthy eye to an artery color deviation degree 4 with low similarity to a healthy eye are used as information indicating a deviation degree from normal artery color, the deviation degree from the normal artery color may be indicated using other arbitrary numbers of labels.

Further, the storage device 30 stores, as third learning data, a fundus image for which information indicating “healthy” or a deviation degree from normal fundus color is labeled as ground truth data. For example, if retinopathy of diabetes progresses, blood components leak or bleeding occurs from blood vessels of the retina, and color of a region other than the blood vessels changes to red compared to color of the normal fundus. While in the present embodiment, a total of three labels from a fundus color deviation degree 1 with high similarity to a healthy eye to a fundus color deviation degree 3 with low similarity to a healthy eye are used as information indicating a deviation degree from normal fundus color, the deviation degree from the normal fundus color may be indicated using other arbitrary numbers of labels.

Here, the deviation degree is not limited to evaluation expressed by, for example, numbers, but includes ranking and other evaluation expressed by characters and symbols such as A, B, C and first, second, third. Note that while in the present embodiment, an example will be described where a fundus image which is one example of a medical image is used, other arbitrary medical images may be used.

Note that while in the present embodiment, an example will be described where a fundus image of a healthy eye and a fundus image of an unhealthy eye in which deviation from a healthy eye can be found are used as learning data to be used for learning of the machine learning model, only a fundus image of a healthy eye may be used as the learning data, or only a fundus image of an unhealthy eye may be used as the learning data.

Further, the storage device 30 stores the learned model output by the learning device 10. While FIG. 1 illustrates the storage device 30 as a single storage device, the storage device 30 may be composed of one or more file servers.

Here, as illustrated in an upper part of FIG. 2, the learning device 10 according to the present embodiment includes a first machine learning model that receives a fundus image as input data and classifies the fundus image into one of an image of a healthy eye and images of unhealthy eyes with the diameter deviation degrees 1 to 4. Further, as illustrated in a lower part of FIG. 2, the learning device 10 includes a second machine learning model that receives a fundus image as input data and classifies the fundus image into one of an image of a healthy eye and images of unhealthy eyes with the artery color deviation degrees 1 to 4. Still further, while not illustrated in FIG. 2, the learning device 10 includes a third machine learning model that receives a fundus image as input data and classifies the fundus image into one of an image of a healthy eye and images of unhealthy eyes with the fundus color deviation degrees 1 to 3. The learning device 10 classifies the fundus image using the machine learning model and causes the machine learning model to learn so that an error between a predicted result and the ground truth data labeled to the learning data becomes minimum.

Note that while in the present embodiment, the machine learning model that also outputs a confidence score of a healthy eye is used, in another embodiment, a machine learning model that does not output a confidence score of a healthy eye and only outputs a confidence score of a certain deviation degree may be used.

Further, while in the present embodiment, classification is performed respectively using the machine learning model for the artery diameter, the machine learning model for the artery color and the machine learning model for the fundus color, in another embodiment, classification of the artery diameter, the artery color and the fundus color may be performed using a single machine learning model.

As illustrated in FIG. 3, the inference device 20 according to the present embodiment performs forward calculation using a diameter learned model and infers which of a healthy eye with a normal artery diameter, an unhealthy eye with the diameter deviation degree 1, an unhealthy eye with the diameter deviation degree 2, an unhealthy eye with the diameter deviation degree 3, and an unhealthy eye with the diameter deviation degree 4, the fundus image belongs to. Then, in a case where the confidence scores of the diameter deviation degrees 1 to 4 are equal to or greater than a certain threshold, the inference device 20 creates a heat map by performing backpropagation from an output layer corresponding to classification having the highest confidence score among the diameter deviation degrees 1 to 4 to a convolutional layer that is desired to be visualized, calculating a contribution degree of each feature map to output of the classification of the diameter deviation degree having the highest confidence score, weighting the feature maps obtained in the forward calculation in accordance with the contribution degree and adding up the feature maps. Note that while in the present embodiment, an example where the heat map is created for the classification of the diameter deviation degree having the highest confidence score is described, in another embodiment, the heat map may be created for classification of all diameter deviation degrees that are equal to or greater than a certain threshold.

Note that while FIG. 3 illustrates the diameter learned model, the inference device 20 performs forward calculation using an artery color learned model in a similar manner and infers which of a healthy eye with normal artery color, an unhealthy eye with the artery color deviation degree 1, an unhealthy eye with the artery color deviation degree 2, an unhealthy eye with the artery color deviation degree 3, and an unhealthy eye with the artery color deviation degree 4, the fundus image belongs to. In a similar manner, in a case where the confidence scores of the artery color deviation degrees 1 to 4 are equal to or greater than a certain threshold, the inference device 20 creates a heat map by performing backpropagation from an output layer corresponding to classification having the highest confidence score among the artery color deviation degrees 1 to 4 to a convolutional layer that is desired to be visualized, calculating a contribution degree of each feature map to output of the classification of the artery color deviation degree having the highest confidence score, weighting the feature maps obtained in the forward calculation in accordance with the contribution degree and adding up the feature maps.

In a similar manner, the inference device 20 performs forward calculation using a fundus color learned model and infers which of a healthy eye with normal fundus color, an unhealthy eye with the fundus color deviation degree 1, an unhealthy eye with the fundus color deviation degree 2, and an unhealthy eye with the fundus color deviation degree 3, the fundus image belongs to. In a similar manner, in a case where the confidence scores of the fundus color deviation degrees 1 to 3 are equal to or greater than a certain threshold, the inference device 20 creates a heat map by performing backpropagation from an output layer corresponding to classification having the highest confidence score among the fundus color deviation degrees 1 to 3 to a convolutional layer that is desired to be visualized, calculating a contribution degree of each feature map to output of the classification of the fundus color deviation degree having the highest confidence score, weighting the feature maps obtained in the forward calculation in accordance with the contribution degree and adding up the feature maps.

By performing inference regarding the artery diameter of the fundus image using the learned model that has learned features of the artery diameter from a plurality of images of healthy eyes and images of unhealthy eyes in which artery diameters deviate from the normal artery diameter, it is possible to comprehensively infer whether or not a plurality of artery diameters included in the image deviate from the normal artery diameter instead of inferring whether or not individual artery diameters are normal.

In a similar manner, by performing inference regarding the artery color of the fundus image using the learned model that has learned features of the artery color from a plurality of images of healthy eyes and images of unhealthy eyes in which artery color deviates from the normal artery color, it is possible to comprehensively infer whether or not color of a plurality of arteries included in the image deviates from normal artery color instead of inferring whether or not color of individual arteries is normal.

In a similar manner, by performing inference regarding the fundus color of the fundus image using the learned model that has learned features of the fundus color from a plurality of images of healthy eyes and images of unhealthy eyes in which fundus color deviates from the normal fundus color, it is possible to comprehensively infer whether or not the fundus color included in the image deviates from the normal fundus color instead of inferring whether or not color of individual fundus regions is normal.

### Functional Configuration: Learning Device

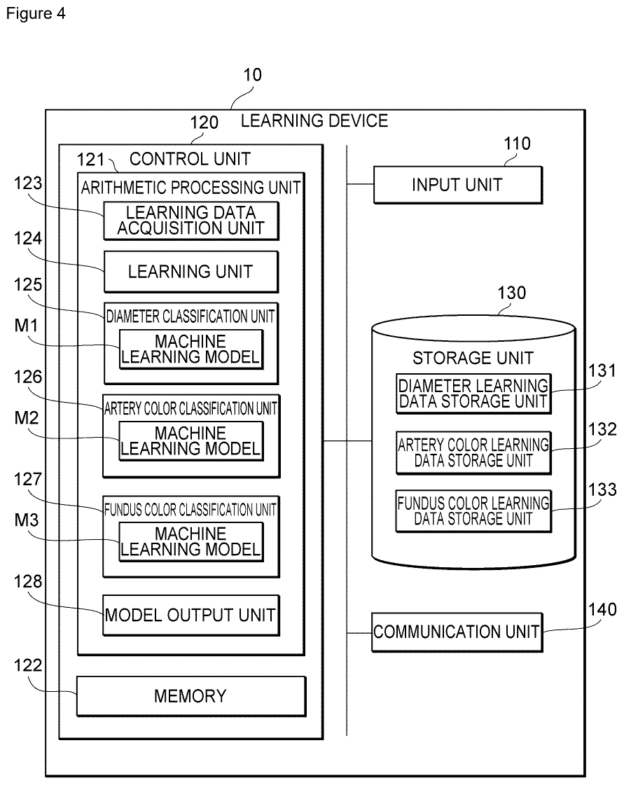

FIG. 4 is a block diagram of a learning device according to an embodiment. Note that while FIG. 4 illustrates only necessary functional components assuming a single learning device 10, the learning device 10 can be constituted as part of a distributed system having multiple functions by a plurality of computer systems.

The learning device 10 includes an input unit 110, a control unit 120, a storage unit 130, and a communication unit 140.

The input unit 110 is configured to accept operation from a manager of the learning device 10 and can be implemented with a keyboard, a mouse, a touch panel, and the like.

The control unit 120 includes an arithmetic processing unit 121 such as a CPU and an MPU corresponding to a processor, and a memory 122 such as a RAM. The arithmetic processing unit 121 (processor) implements functions and processing which will be described later in the arithmetic processing unit 121 by loading a program recorded in the storage unit 130 to the memory 122 and executing the program based on various kinds of inputs. This program may be a program that is stored in a computer-readable non-transitory recording medium such as a CD-ROM or distributed via a network and installed in the computer. The memory 122 functions as a work memory necessary for executing the program by the arithmetic processing unit 121 (processor).

The storage unit 130 is constituted with a storage device such as a hard disk and stores various kinds of programs necessary for executing processing in the control unit 120, data necessary for executing various kinds of programs, and the like. In the present embodiment, the storage unit 130 preferably includes a diameter learning data storage unit 131, an artery color learning data storage unit 132, and a fundus color learning data storage unit 133. Note that while in the present embodiment, an example where a machine learning model is caused to learn using learning data for an artery diameter, learning data for artery color and learning data for fundus color will be described, in another embodiment, the machine learning model may be caused to learn using a single piece of learning data.

The diameter learning data storage unit 131 stores learning data to be used for learning of a machine learning model M1 which will be described later. In the present embodiment, the diameter learning data storage unit 131 stores a fundus image for which information indicating “healthy” or a deviation degree from a normal artery diameter is labeled as ground truth data.

The artery color learning data storage unit 132 stores learning data to be used for learning of a machine learning model M2 which will be described later. In the present embodiment, the artery color learning data storage unit 132 stores a fundus image for which information indicating “healthy” or a deviation degree from normal artery color is labeled as ground truth data.

The fundus color learning data storage unit 133 stores learning data to be used for learning of a machine learning model M3 which will be described later. In the present embodiment, the fundus color learning data storage unit 133 stores a fundus image for which information indicating “healthy” or a deviation degree from normal fundus color is labeled as ground truth data.

The communication unit 140 is configured so as to connect the learning device 10 to a network. For example, the communication unit 140 can be implemented with a LAN card, an analog modem, an ISDN modem, or the like, and an interface for connecting these to the processing unit via a transmission path such as a system bus.

Further, as illustrated in FIG. 4, the arithmetic processing unit 121 includes a learning data acquisition unit 123, a learning unit 124, a diameter classification unit 125, an artery color classification unit 126, a fundus color classification unit 127, and a model output unit 128 as functional units. As described above, while in the present embodiment, an example will be described where the respective classification units perform classification using a machine learning model for an artery diameter, a machine learning model for artery color and a machine learning model for fundus color, in another embodiment, a single classification unit may perform classification of the artery diameter, the artery color and the fundus color using a single machine learning model.

The learning data acquisition unit 123 acquires learning data to be used for learning of the machine learning model M1, the machine learning model M2 and the machine learning model M3, which will be described later, and stores the learning data respectively in the diameter learning data storage unit 131, the artery color learning data storage unit 132 and the fundus color learning data storage unit 133.

In the present embodiment, the learning data acquisition unit 123 acquires a fundus image for which information indicating “healthy” or a deviation degree from the normal artery diameter is labeled as ground truth data from the storage device 30 and stores the fundus image in the diameter learning data storage unit 131.

Further, the learning data acquisition unit 123 acquires a fundus image for which information indicating “healthy” or a deviation degree from the normal artery color is labeled as ground truth data from the storage device 30 and stores the fundus image in the artery color learning data storage unit 132. Still further, the learning data acquisition unit 123 acquires a fundus image for which information indicating “healthy” or a deviation degree from the normal fundus color is labeled as ground truth data from the storage device 30 and stores the fundus image in the fundus color learning data storage unit 133.

The learning unit 124 causes the machine learning model M1, the machine learning model M2 and the machine learning model M3 to learn using the learning data acquired by the learning data acquisition unit 123. In the present embodiment, the learning unit 124 inputs the fundus image for which information indicating “healthy” or a deviation degree from the normal artery diameter is labeled as ground truth data to a diameter classification unit 125 which will be described later to cause the machine learning model M1 to learn. Further, the learning unit 124 inputs a fundus image for which information indicating “healthy” or a deviation degree from the normal artery color is labeled as ground truth data to an artery color classification unit 126 which will be described later to cause the machine learning model M2 to learn. Still further, the learning unit 124 inputs a fundus image for which information indicating “healthy” or a deviation degree from the normal fundus color is labeled as ground truth data to a fundus color classification unit 127 which will be described later to cause the machine learning model M3 to learn.

The diameter classification unit 125 accepts input of a fundus image and classifies the fundus image into one of an image of a healthy eye, an image of an unhealthy eye with the diameter deviation degree 1, an image of an unhealthy eye with the diameter deviation degree 2, an image of an unhealthy eye with the diameter deviation degree 3, and an image of an unhealthy eye with the diameter deviation degree 4. In the present embodiment, the diameter classification unit 125 accepts input of a fundus image and outputs confidence scores of the healthy eye, the diameter deviation degree 1, the diameter deviation degree 2, the diameter deviation degree 3, and the diameter deviation degree 4 using the machine learning model M1.

The machine learning model M1 is a machine learning model that receives an fundus image as input data and classifies the fundus image into one of an image of a healthy eye, an image of an unhealthy eye with the diameter deviation degree 1, an image of an unhealthy eye with the diameter deviation degree 2, an image of an unhealthy eye with the diameter deviation degree 3, and an image of an unhealthy eye with the diameter deviation degree 4. In the present embodiment, an example will be described where a convolutional neural network (CNN) that receives a fundus image as input data and outputs classification of the image is used as one example of the machine learning model M1. However, the CNN is merely one example of the machine learning model M1, and the learning device 10 may use other configurations as the machine learning model M1.

The artery color classification unit 126 accepts input of a fundus image and classifies the fundus image into one of an image of a healthy eye, an image of an unhealthy eye with the artery color deviation degree 1, an image of an unhealthy eye with the artery color deviation degree 2, an image of an unhealthy eye with the artery color deviation degree 3, and an image of an unhealthy eye with the artery color deviation degree 4. In the present embodiment, the artery color classification unit 126 accepts input of a fundus image and outputs confidence scores of the healthy eye, the artery color deviation degree 1, the artery color deviation degree 2, the artery color deviation degree 3, and the artery color deviation degree 4 using the machine learning model M2.

The machine learning model M2 is a machine learning model that receives a fundus image as input data and classifies the fundus image into one of an image of a healthy eye, an image of an unhealthy eye with the artery color deviation degree 1, an image of an unhealthy eye with the artery color deviation degree 2, an image of an unhealthy eye with the artery color deviation degree 3, and an image of an unhealthy eye with the artery color deviation degree 4. In the present embodiment, an example will be described where a convolutional neural network (CNN) that receives a fundus image as input data and outputs classification of the image is used as one example of the machine learning model M2. However, the CNN is merely one example of the machine learning model M2, and the learning device 10 may use other configurations as the machine learning model M2.

The fundus color classification unit 127 accepts input of a fundus image and classifies the fundus image into one of an image of a healthy eye, an image of an unhealthy eye with the fundus color deviation degree 1, an image of an unhealthy eye with the fundus color deviation degree 2, and an image of an unhealthy eye with the fundus color deviation degree 3. In the present embodiment, the fundus color classification unit 127 accepts input of a fundus image and outputs confidence scores of the healthy eye, the fundus color deviation degree 1, the fundus color deviation degree 2, and the fundus color deviation degree 3 using the machine learning model M3.

The machine learning model M3 is a machine learning model that receives a fundus image as input data and classifies the fundus image into one of an image of a healthy eye, an image of an unhealthy eye with the fundus color deviation degree 1, an image of an unhealthy eye with the fundus color deviation degree 2, and an image of an unhealthy eye with the fundus color deviation degree 3. In the present embodiment, an example will be described where a convolutional neural network (CNN) that receives a fundus image as input data and outputs classification of the image is used as one example of the machine learning model M3. However, the CNN is merely one example of the machine learning model M3, and the learning device 10 may use other configurations as the machine learning model M3.

The learning unit 124 causes each of the machine learning model M1, and the machine learning models M2 and M3 to learn so that an error between results predicted by the machine learning model M1, and the machine learning models M2 and M3, and the ground truth data labeled to the learning data becomes minimum.

When learning of the machine learning model M1 is completed, the model output unit 128 outputs the diameter learned model obtained through learning of the machine learning model M1 to the storage device 30. Further, when learning of the machine learning model M2 is completed, the model output unit 128 outputs the artery color learned model obtained through learning of the machine learning model M2 to the storage device 30. Still further, when learning of the machine learning model M3 is completed, the model output unit 128 outputs the artery color learned model obtained through learning of the machine learning model M3 to the storage device 30. Note that the learning unit 124 may complete learning, for example, after causing the machine learning model to learn using a predetermined number of pieces of learning data or may complete learning in a case where accuracy of classification predicted using the machine learning model satisfies a certain condition.

### Functional Configuration: Inference Device

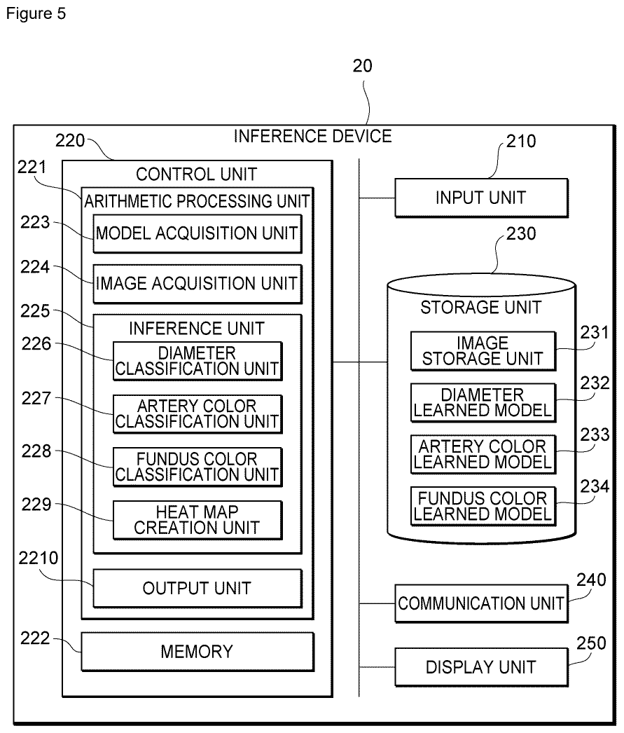

FIG. 5 is a block diagram of an inference device according to an embodiment. Note that while FIG. 5 illustrates only necessary functional components assuming a single inference device 20, the inference device 20 may be constituted as part of a distributed system having multiple functions by a plurality of computer systems, or part of the functional components in the inference device 20 may be implemented with cloud computing including one or more information processing devices. For example, a computer or a fundus camera can be used as the inference device 20.

The inference device 20 includes an input unit 210, a control unit 220, a storage unit 230, a communication unit 240, and a display unit 250.

The input unit 210 is configured to accept operation from a manager of the inference device 20 and can be implemented with a keyboard, a mouse, a touch panel, or the like.

The control unit 220 includes an arithmetic processing unit 221 such as a CPU and an MPU corresponding to a processor, and a memory 222 such as a RAM. The arithmetic processing unit 221 (processor) implements functions and processing which will be described later in the arithmetic processing unit 221 by loading a program recorded in the storage unit 230 to the memory 222 and executing the program based on various kinds of inputs. This program may be a program that is stored in a computer-readable non-transitory recording medium such as a CD-ROM or distributed via a network and installed in a computer. The memory 222 functions as a work memory necessary for executing the program by the arithmetic processing unit 221 (processor).

The storage unit 230 is constituted with a storage device such as a hard disk and records various kinds of programs necessary for executing processing in the control unit 220, data necessary for executing various kinds of programs, and the like. In the present embodiment, the storage unit 230 preferably includes an image storage unit 231, a diameter learned model 232, an artery color learned model 233, and a fundus color learned model 234. As described above, while in the present embodiment, an example will be described where respective classification units perform classification using the learned model for the artery diameter, the learned model for the artery color, and the learned model for the fundus color, in another embodiment, a single classification unit may perform classification of the artery diameter, the artery color and the fundus color using a single learned model.

The image storage unit 231 stores an image for which inference is to be performed. In the present embodiment, the image storage unit 231 stores a fundus image for which the diameter deviation degree, the artery color deviation degree, and the fundus color deviation degree are inferred.

The diameter learned model 232 stores a learned model to be used for inference. In the present embodiment, the diameter learned model 232 stores a learned model that receives a fundus image as input data and classifies the fundus image into one of an image of a healthy eye, an image of an unhealthy eye with the diameter deviation degree 1, an image of an unhealthy eye with the diameter deviation degree 2, an image of an unhealthy eye with the diameter deviation degree 3, and an image of an unhealthy eye with the diameter deviation degree 4. In the present embodiment, an example will be described where a convolutional neural network (CNN) that receives a fundus image as input data and classifies the fundus image into one of an image of a healthy eye, an image of an unhealthy eye with the diameter deviation degree 1, an image of an unhealthy eye with the diameter deviation degree 2, an image of an unhealthy eye with the diameter deviation degree 3, and an image of an unhealthy eye with the diameter deviation degree 4 is used as one example of the diameter learned model 232. However, the CNN is merely one example of the diameter learned model 232, and the inference device 20 may use other configurations as the diameter learned model 232.

The artery color learned model 233 stores a learned model to be used for inference. In the present embodiment, the artery color learned model 233 stores a learned model that receives a fundus image as input data and classifies the fundus image into one of an image of a healthy eye, an image of an unhealthy eye with the artery color deviation degree 1, an image of an unhealthy eye with the artery color deviation degree 2, an image of an unhealthy eye with the artery color deviation degree 3, and an image of an unhealthy eye with the artery color deviation degree 4. In the present embodiment, an example will be described where a convolutional neural network (CNN) that receives a fundus image as input data and classifies the fundus image into one of an image of a healthy eye, an image of an unhealthy eye with the artery color deviation degree 1, an image of an unhealthy eye with the artery color deviation degree 2, an image of an unhealthy eye with the artery color deviation degree 3, and an image of an unhealthy eye with the artery color deviation degree 4 is used as one example of the artery color learned model 233. However, the CNN is merely one example of the artery color learned model 233, and the inference device 20 may use other configurations as the artery color learned model 233.

The fundus color learned model 234 stores a learned model to be used for inference. In the present embodiment, the fundus color learned model 234 stores a learned model that receives a fundus image as input data and classifies the fundus image into one of an image of a healthy eye, an image of an unhealthy eye with the fundus color deviation degree 1, an image of an unhealthy eye with the fundus color deviation degree 2, and an image of an unhealthy eye with the fundus color deviation degree 3. In the present embodiment, an example will be described where a convolutional neural network (CNN) that receives a fundus image as input data and classifies the fundus image into one of an image of a healthy eye, an image of an unhealthy eye with the fundus color deviation degree 1, an image of an unhealthy eye with the fundus color deviation degree 2, and an image of an unhealthy eye with the fundus color deviation degree 3 is used as one example of the fundus color learned model 234. However, the CNN is merely one example of the fundus color learned model 234, and the inference device 20 may use other configurations as the fundus color learned model 234.

The communication unit 240 is configured to connect the inference device 20 to a network. For example, the communication unit 240 can be implemented with a LAN card, an analog modem, an ISDN modem, or the like, and an interface for connecting these to a processing unit via a transmission path such as a system bus.

The display unit 250 is a device for displaying information and can be implemented with an organic EL display, a liquid crystal display, or the like.

Further, as illustrated in FIG. 5, the arithmetic processing unit 221 includes a model acquisition unit 223, an image acquisition unit 224, an inference unit 225, a diameter classification unit 226, an artery color classification unit 227, a fundus color classification unit 228, a heat map creation unit 229, and an output unit 2210 as functional units.

The model acquisition unit 223 acquires a learned model to be used for inference and stores the learned model in the storage unit 230. In the present embodiment, the model acquisition unit 223 acquires the diameter learned model from the storage device 30 and stores the diameter learned model in the diameter learned model 232. Further, the model acquisition unit 223 acquires the artery color learned model from the storage device 30 and stores the artery color learned model in the artery color learned model 233. Still further, the model acquisition unit 223 acquires the fundus color learned model from the storage device 30 and stores the fundus color learned model in the fundus color learned model 234.

The image acquisition unit 224 acquires an image for which inference is to be performed. In the present embodiment, the image acquisition unit 224 acquires a fundus image for which inference is to be performed from the image storage unit 231.

The inference unit 225 infers a deviation degree from a healthy eye, predicted from the image acquired by the image acquisition unit 224. In the present embodiment, the inference unit 225 is constituted with the diameter classification unit 226, the artery color classification unit 227, the fundus color classification unit 228, and the heat map creation unit 229. The inference unit 225 first inputs a fundus image to the diameter classification unit 226 and acquires confidence scores of the healthy eye, the diameter deviation degree 1, the diameter deviation degree 2, the diameter deviation degree 3, and the diameter deviation degree 4 from the diameter classification unit 226.

The diameter classification unit 226 accepts input of a fundus image and classifies the fundus image into one of an image of a healthy eye, an image of an unhealthy eye with the diameter deviation degree 1, an image of an unhealthy eye with the diameter deviation degree 2, an image of an unhealthy eye with the diameter deviation degree 3, and an image of an unhealthy eye with the diameter deviation degree 4. As illustrated in FIG. 3, in the present embodiment, the diameter classification unit 226 accepts input of a fundus image, performs forward calculation using the diameter learned model 232 and outputs confidence scores of the healthy eye, the diameter deviation degree 1, the diameter deviation degree 2, the diameter deviation degree 3, and the diameter deviation degree 4.

Further, the inference unit 225 inputs the fundus image to the artery color classification unit 227 and acquires confidence scores of the healthy eye, the artery color deviation degree 1, the artery color deviation degree 2, the artery color deviation degree 3, the artery color deviation degree 4 from the artery color classification unit 227. In the present embodiment, the artery color classification unit 227 accepts input of the fundus image, performs forward calculation using the artery color learned model 233 and outputs confidence scores of the healthy eye, the artery color deviation degree 1, the artery color deviation degree 2, the artery color deviation degree 3, and the artery color deviation degree 4.

Further, the inference unit 225 inputs the fundus image to the fundus color classification unit 228 and acquires confidence scores of the healthy eye, the fundus color deviation degree 1, the fundus color deviation degree 2, and the fundus color deviation degree 3 from the fundus color classification unit 228. In the present embodiment, the fundus color classification unit 228 accepts input of the fundus image, performs forward calculation using the fundus color learned model 234 and outputs confidence scores of the healthy eye, the fundus color deviation degree 1, the fundus color deviation degree 2, and the fundus color deviation degree 3.

Note that while in the present embodiment, an example will be described where the fundus color classification unit 228 outputs the confidence scores of the healthy eye, the fundus color deviation degree 1, the fundus color deviation degree 2, and the fundus color deviation degree 3 using the fundus color learned model 234, in another example, the fundus color classification unit 228 may identify a region in red color other than blood vessels in the fundus image without using the learned model and may output the confidence scores of the healthy eye, the fundus color deviation degree 1, the fundus color deviation degree 2, and the fundus color deviation degree 3 based on an area, a position, and the like, of the identified region in red color.

The heat map creation unit 229 creates a heat map in which a region that contributes to classification by the diameter classification unit 226 is visualized. As illustrated in FIG. 3, in the present embodiment, when the healthy eye, the diameter deviation degree 1, the diameter deviation degree 2, the diameter deviation degree 3, and the diameter deviation degree 4 are output, the heat map creation unit 229 performs backpropagation from an output layer corresponding to classification having the highest confidence score among the diameter deviation degrees 1 to 4 to a convolutional layer that is desired to be visualized, calculates a gradient of a feature map for output of target classification of the diameter deviation degree to calculate a contribution degree of each feature map to output of the classification of the diameter deviation degree having the highest confidence score, and takes global max pooling (GMP) of the gradient. Next, the heat map creation unit 229 weights the feature maps obtained in the forward calculation in accordance with the GMP and acquires a coefficient map by adding all the feature maps.

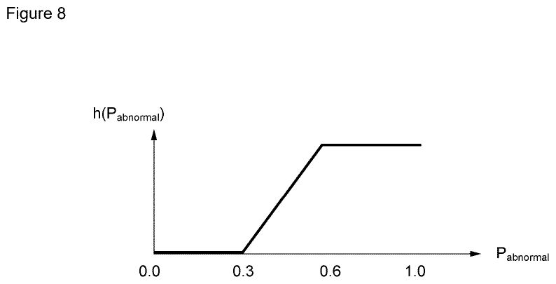

In an embodiment, the heat map creation unit 229 adjusts values of respective elements of the coefficient map based on a confidence score of the target classification of the diameter deviation degree. For example, as indicated in FIG. 8, the heat map creation unit 229 may set the values of the respective elements of the coefficient map at zero in a case where the confidence score of the target classification of the diameter deviation degree is from 0.0 to 0.3, adjust the values of the respective elements in proportion to the confidence score of the target classification of the diameter deviation degree in a case where the confidence score of the target classification of the diameter deviation degree is from 0.3 to 0.6, and adjust the values of the respective elements at 100%, that is, the values as they are in a case where the confidence score of the target classification of the diameter deviation degree is from 0.6 to 1.0. By decreasing the values of the respective elements of the coefficient map in a case where the confidence score of the target classification of the diameter deviation degree is low, and maintaining the values of the respective elements of the coefficient map in a case where the confidence score is high, it is possible to point out, with high accuracy, the region in which deviation from the healthy eye is anticipated.

Finally, the heat map creation unit 229 creates a heat map by creating an image of the coefficient map with color scale and resizing the obtained image to a size of an input image.

While in the present embodiment, an example will be described where a heat map is created through gradient-weighted class activation mapping (Grad-CAM), the heat map may be acquired using other visualization methods. Further, while in the present embodiment, a heat map that expresses a level of the contribution degree with color scale is created, a visualized map that expresses a level of the contribution degree in other formats may be created.

Further, the heat map creation unit 229 creates a heat map in which a region that contributes to classification by the artery color classification unit 227 is visualized in a similar manner to the heat map in which the region that contributes to classification by the diameter classification unit 226 is visualized. Processing of creating a heat map in which the region that contributes to the classification by the artery color classification unit 227 is visualized is similar to processing of creating a heat map in which the region that contributes to the classification by the diameter classification unit 226 is visualized, and thus, description will be omitted.

Further, the heat map creation unit 229 creates a heat map in which a region that contributes to classification by the fundus color classification unit 228 is visualized in a similar manner to the heat map in which the region that contributes to the classification by the diameter classification unit 226 is visualized. Processing of creating a heat map in which the region that contributes to the classification by the fundus color classification unit 228 is visualized is also similar to processing of creating a heat map in which the region that contributes to the classification by the diameter classification unit 226 is visualized, and thus, description will be omitted.

The output unit 2210 outputs the inference result based on the information acquired by the inference unit 225. In the present embodiment, the output unit 2210 outputs information regarding confidence scores of the healthy eye, the diameter deviation degree 1, the diameter deviation degree 2, the diameter deviation degree 3, and the diameter deviation degree 4 acquired by the diameter classification unit 226, information regarding confidence scores of the healthy eye, the artery color deviation degree 1, the artery color deviation degree 2, the artery color deviation degree 3, and the artery color deviation degree 4 acquired by the artery color classification unit 227, and information regarding confidence scores of the healthy eye, the fundus color deviation degree 1, the fundus color deviation degree 2, and the fundus color deviation degree 3 acquired by the fundus color classification unit 228. Further, the output unit 2210 can superimpose the heat map in which the region that contributes to the classification by the diameter classification unit 226 is visualized, the heat map in which the region that contributes to the classification by the artery color classification unit 227 is visualized, or the heat map in which the region that contributes to the classification by the fundus color classification unit 228 is visualized on the fundus image acquired by the image acquisition unit 224 by alpha blending, and output the superimposed result.

### Learning Processing

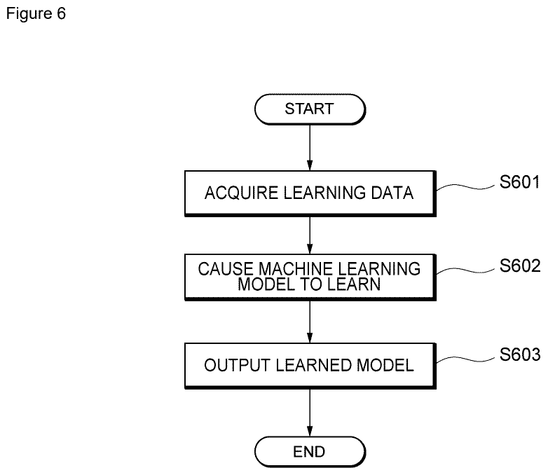

Learning processing by the learning device according to an embodiment will be described in detail with reference to FIG. 6. It is assumed in the present embodiment that before the learning processing described with FIG. 6 is performed, learning data is stored in the storage device 30 under the supervision of the manager of the learning device 10. Note that the processing indicated in FIG. 6 is executed, for example, by the manager inputting an instruction to execute processing of generating a learned model via the input unit 110. Note that processing of generating a diameter learned model will be described here. Processing of generating an artery color learned model and a fundus color learned model is similar to the processing of generating a diameter learned model, and thus, description will be omitted.

In step S601, the learning data acquisition unit 123 of the learning device 10 acquires learning data to be used for learning of the machine learning model M and stores the learning data in the diameter learning data storage unit 131. In the present embodiment, the learning data acquisition unit 123 acquires a fundus image for which information indicating “healthy” or a deviation degree from the normal artery diameter is labeled as ground truth data from the storage device 30 and stores the fundus image in the diameter learning data storage unit 131.

Then, in step S602, the learning unit 124 of the learning device 10 causes the machine learning model M1 to learn using the learning data acquired by the learning data acquisition unit 123. In the present embodiment, the learning unit 124 inputs the fundus image for which information indicating “healthy” or a deviation degree from the normal artery diameter is labeled as ground truth data to the diameter classification unit 125 of the learning device 10 to cause the machine learning model M1 to learn.

The machine learning model M1 is a machine learning model that receives a fundus image as input data and classifies the fundus image into one of an image of a healthy eye, an image of an unhealthy eye with the diameter deviation degree 1, an image of an unhealthy eye with the diameter deviation degree 2, an image of an unhealthy eye with the diameter deviation degree 3, and an image of an unhealthy eye with the diameter deviation degree 4. In the present embodiment, an example will be described where a convolutional neural network (CNN) that receives a fundus image as input data and outputs the classification of the image is used as one example of the machine learning model M1. However, the CNN is merely one example of the machine learning model M1, and the learning device 10 may use other configurations as the machine learning model M1.

The learning unit 124 causes the machine learning model M1 to learn so that an error between a result predicted by the machine learning model M1 and the ground truth data labeled to the learning data becomes minimum.

When learning of the machine learning model M1 is completed, in step S603, the model output unit 128 of the learning device 10 outputs the diameter learned model obtained through learning of the machine learning model M1 to the storage device 30. Note that the learning unit 124 may complete learning, for example, after causing the machine learning model M1 to learn using a predetermined number of pieces of learning data or may complete learning in a case where accuracy of classification predicted using the machine learning model M1 satisfies a certain condition.

### Inference Processing

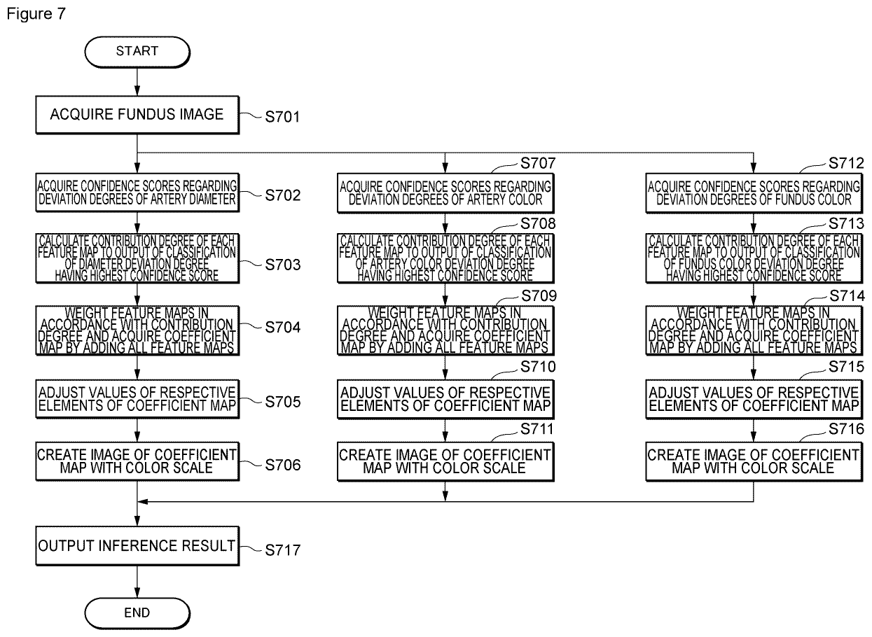

Inference processing by the inference device according to an embodiment will be described in detail with reference to FIG. 7. It is assumed in the present embodiment that before the inference processing described with FIG. 7 is performed, the learned models acquired from the storage device 30 are stored in the diameter learned model 232, the artery color learned model 233, and the fundus color learned model 234 under the supervision of the manager of the inference device 20. Further, it is assumed that a fundus image for which inference is to be performed, acquired by imaging of the fundus that is a portion to be examined of the subject, is stored in the image storage unit 231 of the inference device 20. Note that the processing indicated in FIG. 7 is executed, for example, by the manager inputting an instruction to execute the inference processing via the input unit 210.

In step S701, the image acquisition unit 224 of the inference device 20 acquires an image for which inference is to be performed. In the present embodiment, the image acquisition unit 224 acquires a fundus image for which inference of the diameter deviation degree, the artery color deviation degree, and the fundus color deviation degree is to be performed from the image storage unit 231.

In step S702, the inference unit 225 of the inference device 20 acquires a confidence score regarding the deviation degree of the artery diameter. In the present embodiment, the inference unit 225 acquires confidence scores of the healthy eye, the diameter deviation degree 1, the diameter deviation degree 2, the diameter deviation degree 3, and the diameter deviation degree 4 from the diameter classification unit 226 by inputting the fundus image to the diameter classification unit 226 of the inference device 20. Specifically, the diameter classification unit 226 accepts input of a fundus image, performs forward calculation using the diameter learned model 232, and outputs the confidence scores of the healthy eye, the diameter deviation degree 1, the diameter deviation degree 2, the diameter deviation degree 3, and the diameter deviation degree 4.

In the present embodiment, an example will be described where a convolutional neural network (CNN) that receives a fundus image as input data and classifies the fundus image into one of an image of a healthy eye, an image of an unhealthy eye with the diameter deviation degree 1, an image of an unhealthy eye with the diameter deviation degree 2, an image of an unhealthy eye with the diameter deviation degree 3, and an image of an unhealthy eye with the diameter deviation degree 4 is used as one example of the diameter learned model 232. However, the CNN is merely one example of the diameter learned model 232, and the inference device 20 may use other configurations as the diameter learned model 232.

In a case where the confidence scores regarding the deviation degrees of the artery diameter are output, and the confidence scores of the diameter deviation degrees 1 to 4 are equal to or greater than a certain threshold, in step S703, the heat map creation unit 229 of the inference device 20 performs backpropagation from an output layer corresponding to classification having the highest confidence score among the diameter deviation degrees 1 to 4 to a convolutional layer that is desired to be visualized, calculates a gradient of a feature map for output of the target classification of the diameter deviation degree to calculate a contribution degree of each feature map to output of the classification of the diameter deviation degree having the highest confidence score, and takes global max pooling (GMP) of the gradient. In a case where the confidence scores of the diameter deviation degrees 1 to 4 are less than the certain threshold, the processing from step S703 to step S706 is skipped, and the processing proceeds to S717.

In step S704, the heat map creation unit 229 weights the feature maps obtained in the forward calculation in accordance with the GMP and acquires a coefficient map by adding all the feature maps. Then, in step S705, the heat map creation unit 229 adjusts values of the respective elements of the coefficient map based on the confidence scores of the unhealthy eyes.

As indicated in FIG. 8, in the present embodiment, the heat map creation unit 229 may set the values of the respective elements of the coefficient map at zero in a case where the confidence score of the target classification of the diameter deviation degree is from 0.0 to 0.3, adjust the values of the respective elements in proportion to the confidence score of the target classification of the diameter deviation degree in a case where the confidence score of the target classification of the diameter deviation degree is from 0.3 to 0.6, and adjust the values of the respective elements to 100%, that is, the values are they are, in a case where the confidence score of the target classification of the diameter deviation degree is from 0.6 to 1.0. By decreasing the values of the respective elements of the coefficient map in a case where the confidence score of the target classification of the diameter deviation degree is low and maintaining the values of the respective elements of the coefficient map in a case where the confidence score is high, it is possible to point out, with high accuracy, a region where deviation from the healthy eye is anticipated.

Further, in step S706, the heat map creation unit 229 creates a heat map by creating an image of the adjusted coefficient map with color scale and resizing the obtained image to a size of an input image.

Further, after the image for which inference is to be performed is acquired in step S701, the inference unit 225 acquires the confidence scores regarding the deviation degrees of the artery color in parallel to the processing in step S702 (step S707). Note that processing from step S707 to step S711 is similar to the processing from step S702 to step S706, and thus, description will be omitted.

Further, while in the present embodiment, the processing in step S702 and the processing in step S707 are executed in parallel, in another embodiment, the processing from step S707 to step S711 may be executed after the processing from step S702 to step S706, or the processing from step S702 to step S706 may be executed after the processing from step S707 to step S711.

Further, after the image for which inference is to be performed is acquired in step S701, the inference unit 225 acquires the confidence scores regarding the deviation degrees of the fundus color in parallel to the processing in step S702 (step S712). Note that processing from step S712 to step S716 is similar to the processing from step S702 to step S706, and thus, description will be omitted.

Further, while in the present embodiment, the processing in step S702 and the processing in step S712 are executed in parallel, in another embodiment, the processing from step S712 to step S716 may be executed after the processing from step S707 to step S711, or the processing from step S702 to step S706 and the processing from step S707 to step S711 may be executed after the processing from step S712 to step S716.

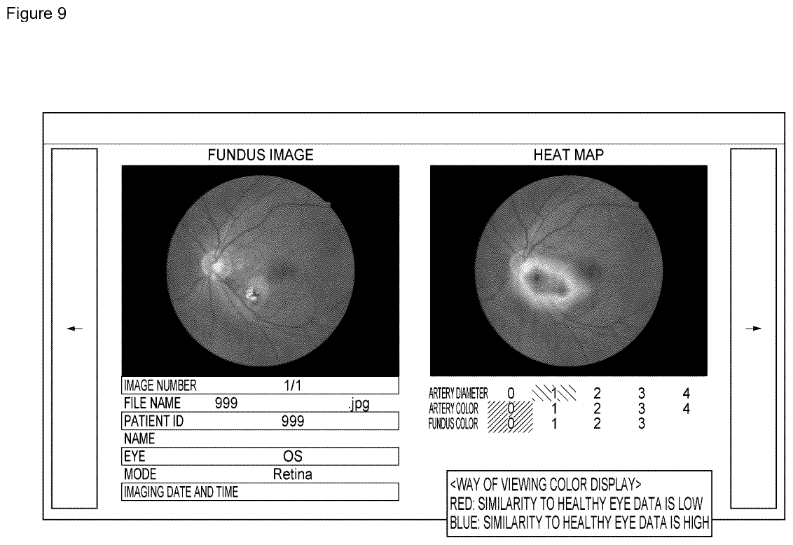

In step S717, the output unit 2210 of the inference device 20 outputs an inference result based on the information acquired by the inference unit 225. In the present embodiment, the output unit 2210 outputs information regarding the confidence scores of the healthy eye, the diameter deviation degree 1, the diameter deviation degree 2, the diameter deviation degree 3, and the diameter deviation degree 4 acquired by the diameter classification unit 226 and information regarding the confidence scores of the healthy eye, the artery color deviation degree 1, the artery color deviation degree 2, the artery color deviation degree 3, and the artery color deviation degree 4 acquired by the artery color classification unit 227. In one embodiment, the output unit 2210 can output information including confidence scores of all classifications acquired by the diameter classification unit 226 and the artery color classification unit 227. Further, as illustrated in FIG. 9, the output unit 2210 superimposes the heat map in which the region that contributes to the classification by the diameter classification unit 226 is visualized or the heat map in which the region that contributes to the classification by the artery color classification unit 227 is visualized on the fundus image acquired by the image acquisition unit 224 by alpha blending, and outputs the superimposed result.

Note that while in FIG. 9, the output unit 2210 also outputs information on the classification having the highest confidence score based on the confidence scores acquired by the respective classification units, in another embodiment, the output unit 2210 may output the confidence scores of all classifications acquired by the respective classification units.

As described above, according to the present embodiment, the learning device 10 can obtain a learned model that can be used for inference of the deviation degrees from the normal artery diameter and/or the normal artery color and/or the normal fundus color.

Further, according to the present embodiment, the inference device 20 can obtain a measurement value obtained by evaluating features of the whole arteries and/or the whole fundus of the fundus image from an image of a healthy eye and images of unhealthy eyes using learned models that have learned features that deviate from the normal artery diameter and/or the normal artery color and/or the normal fundus color. Compared to a case where individual artery diameters, color of the arteries, and color of the fundus are measured and evaluated, the inference device 20 can obtain the measurement result with fewer computer resources.

Further, the inference device 20 can point out, with high accuracy, the region where deviation from the healthy eye is anticipated by visualizing the region that greatly contributes to output of the classification having the highest confidence score among the classifications by the diameter classification unit 226 and/or the artery color classification unit 227 and/or the fundus color classification unit 228 indicating the deviation degrees from the normal artery.

Note that while in the present embodiment, the inference device 20 that infers the diameter deviation degree, the artery color deviation degree and the fundus color deviation degree has been described, only the diameter deviation degree, or only the artery color deviation degree, or only the fundus color deviation degree may be inferred.

Further, while in the present embodiment, an example has been described where a measurement value indicating an evaluation of features of a dimeter of the artery, color of the artery and color of the fundus other than blood vessels is obtained as attributes of a portion to be examined of the fundus image, a measurement value indicating an evaluation of features of other arbitrary attributes such as depression of optic papilla can be obtained. Further, while in the present embodiment, an example has been described where a measurement value indicating an evaluation of particularly a region of red color other than the blood vessels is obtained as features of color of the fundus related to bleeding, a measurement value indicating an evaluation of regions of other colors may be obtained as the features of color of the fundus, or a measurement value indicating an evaluation of features of color of, for example, optic papilla, instead of the whole fundus, may be obtained.

### REFERENCE SIGNS LIST

10 Learning device
110 Input unit
120 Control unit
121 Arithmetic processing unit
122 Memory
123 Learning data acquisition unit
124 Learning unit
125 Diameter classification unit
126 Artery color classification unit
127 Fundus color classification unit
128 Model output unit
130 Storage unit
131 Diameter learning data storage unit
132 Artery color learning data storage unit
133 Fundus color learning data storage unit
140 Communication unit
20 Inference device
210 Input unit
220 Control unit
221 Arithmetic processing unit
222 Memory
223 Model acquisition unit
224 Image acquisition unit
225 Inference unit
226 Diameter classification unit (first classification unit)
227 Artery color classification unit (second classification unit)
228 Fundus color classification unit (third classification unit)
229 Heat map creation unit (image creation unit)
2210 Output unit
230 Storage unit
231 Image storage unit
232 Diameter learned model (first inference model)
233 Artery color learned model (second inference model)
234 Fundus color learned model
240 Communication unit
250 Display unit
30 Storage device
M1 Machine learning model
M2 Machine learning model
M3 Machine learning model
N Communication network

1. An information processing device comprising:
an image acquisition unit that acquires a first image acquired by imaging of a portion to be examined of a subject;
a first classification unit that acquires a confidence score that a first attribute of the portion to be examined of the first image is normal and/or a confidence score that the first attribute of the portion to be examined of the first image has a certain deviation degree from the normal first attribute, the first attribute being an artery diameter; and
an output unit that outputs an inference result based on the confidence score acquired by the first classification unit.

2. The information processing device according to claim 1, further comprising:
a second classification unit that acquires a confidence score that a second attribute of the portion to be examined of the first image is normal and/or a confidence score that the second attribute of the portion to be examined of the first image has a certain deviation degree from the normal second attribute,
wherein the output unit outputs the inference result based on the confidence score acquired by the second classification unit.

3. The information processing device according to claim 2, wherein
the first image is a fundus image of the subject, and
the first attribute and the second attribute are attributes regarding an artery.

4. The information processing device according to claim 2, further comprising:
a third classification unit that acquires a confidence score that a third attribute of the portion to be examined of the first image is normal and/or a confidence score that the third attribute of the portion to be examined of the first image has a certain deviation degree from the normal third attribute,
wherein the output unit outputs the inference result based on the confidence score acquired by the third classification unit.

5. The information processing device according to claim 4, wherein
the first image is a fundus image of the subject,
the first attribute and the second attribute are attributes regarding an artery, and
the third attribute is an attribute regarding a fundus.

6. The information processing device according to claim 4,
wherein the first classification unit, the second classification unit, and the third classification unit are a single classification unit.

7. The information processing device according to claim 2, wherein
the second classification unit acquires the confidence score that the second attribute of the portion to be examined is normal and/or the confidence score that the second attribute of the portion to be examined has a certain deviation degree from the normal second attribute by inputting the first image to a second inference model, and
the second inference model is a model that estimates whether the second attribute of the portion to be examined included in the first image is normal or has a certain deviation degree from the normal second attribute.

8. The information processing device according to claim 1, wherein
the first classification unit acquires the confidence score that the first attribute of the portion to be examined is normal and/or the confidence score that the first attribute of the portion to be examined has a certain deviation degree from the normal first attribute by inputting the first image to a first inference model, and
the first inference model is a model that estimates whether the first attribute of the portion to be examined included in the first image is normal or has a certain deviation degree from the normal first attribute.

9. The information processing device according to claim 1, further comprising:
an image creation unit that creates a second image in which a region that contributes to classification by the first classification unit is visualized,
wherein the output unit outputs the inference result based on the second image created by the image creation unit.

10. The information processing device according to claim 9,
wherein the image creation unit creates the second image by adjusting a value representative of a degree of contribution to the classification by the first classification unit based on a confidence score of the classification by the first classification unit.

11. The information processing device according to claim 1,
wherein the first classification unit acquires a confidence score that the first attribute of the portion to be examined is normal and a plurality of confidence scores that the first attribute of the portion to be examined have certain deviation degrees from the normal first attribute.

12. A method comprising:
acquiring a first image acquired by imaging of a portion to be examined of a subject;
acquiring a confidence score that an artery diameter of the portion to be examined of the first image is normal and/or a confidence score that the artery diameter of the portion to be examined of the first image has a certain deviation degree from the normal artery diameter; and
outputting an inference result based on the confidence score.

13. A computer-readable recording medium recording a program that causes one or more computers to execute:
processing of acquiring a first image acquired by imaging of a portion to be examined of a subject;
processing of acquiring a confidence score that an artery diameter of the portion to be examined of the first image is normal and/or a confidence score that the artery diameter of the portion to be examined of the first image has a certain deviation degree from the normal artery diameter; and
processing of outputting an inference result based on the confidence score.

14. An information processing device comprising:
a learning unit that causes a machine learning model to learn by inputting, to the machine learning model, an image for which information indicating healthy or a deviation degree from a normal artery diameter is labeled as ground truth data; and
a model output unit that outputs the learned model learned at the learning unit.
